(19) United States
(12) Patent Application Publication
(10) Pub. No.: US 2025/0152409 A1
(43) Pub. Date: May 15, 2025

(54) Heating or Cooling Eyepatch Device

(71) Applicant: Jody Seida, Fowler, MI (US)

(72) Inventor: Jody Seida, Fowler, MI (US)

(21) Appl. No.: 18/917,253

(22) Filed: Oct. 16, 2024

Related U.S. Application Data
(60) Provisional application No. 63/598,728, filed on Nov. 14, 2023.

(51) Int. Cl. A61F 7/02; A61F 7/00; A61F 9/04

(52) U.S. Cl. CPC A61F 7/02; A61F 9/04; A61F 2007/0004; A61F 2007/0226

(57) ABSTRACT

A heating or cooling eyepatch device is provided. The device is comprised of an eyepatch body with a pocket to receive an insert that can be chilled or heated, providing a hands-free method for applying temperature therapy to the eye area. The eyepatch body can be made from various materials, including antimicrobial fabrics, and features a pocket accessible via different types of fasteners. Inserts are designed to match the body's shape and may contain a cooling gel mixture or a heat-retaining medium. The eyepatch can be secured to the user's face with either a skin-safe adhesive fastener or an adjustable strap. Two methods of use involve cooling or heating the insert, placing it in the pocket, and securing the eyepatch to the face to deliver the therapeutic effect.

### CROSS-REFERENCE TO RELATED APPLICATION

The present application claims priority to, and the benefit of, U.S. Provisional Application No. 63/598,728, which was filed on Nov. 11, 2023, and is incorporated herein by reference in its entirety.

### FIELD OF THE INVENTION

The present invention relates generally to the field of eyepatches. More specifically, the present invention relates to a heating or cooling eyepatch device that provides an eyepatch with a pocket that receives a removable heating or cooling insert. The eyepatch body may attach to a user's eye area via a fastener such as an adhesive or around a user's head via a strap. Accordingly, the present disclosure makes specific reference thereto. Nonetheless, it is to be appreciated that aspects of the present invention are also equally applicable to other like applications, devices, and methods of manufacture.

### BACKGROUND

Without the application of cold or warm compresses, swelling around the eye may continue unabated or even worsen over time. Such swelling can lead to significant discomfort, and pain, and can severely interfere with one's ability to see clearly. The absence of the soothing effects of these compresses means that the pain and discomfort typically associated with an eye injury or infection could not only persist but potentially intensify as well. Specifically, in the case of an eye injury, opting not to use ice can lead to more pronounced bruising or a more severe black eye. This is because cold compresses play a crucial role in minimizing the extent of discoloration by reducing underlying blood flow to the affected area. Moreover, both cold and warm compresses typically require manual application, where the individual must hold the pack directly over the affected eye, maintaining consistent pressure and positioning to achieve the desired therapeutic effect. This manual application is essential to ensure the compress adequately covers the area, providing relief and promoting healing. In contrast, head-worn heating or cooling packs are designed to cover both eyes simultaneously, offering a more general treatment that might not focus specifically on the injured or infected area but provides broader soothing effects that are undesired. Similarly, infections may take longer to resolve without the appropriate application of compresses, potentially prolonging the overall recovery process if left untreated. Such prolonged inflammation and discomfort can have a negative impact on one's overall well-being and quality of life.

Therefore, there exists a long-felt need in the art for an improved heating and cooling device for application to a user's eye area. There also exists a long-felt need in the art for a heating or cooling eyepatch device that provides heating or cooling relief to a user's eye area. More specifically, there exists a long-felt need in the art for a heating or cooling eyepatch device that provides heating or cooling relief to a user's eye area without the need to manually hold the device on said eye area. In addition, there exists a long-felt need in the art for a heating or cooling eyepatch device that provides heating or cooling relief to a user's eye area without the need to manually hold the device on said eye area, wherein the device only covers one eye of the user during use.

The subject matter disclosed and claimed herein, in one embodiment thereof, comprises a heating or cooling eyepatch device. The device is comprised of an eyepatch body with a pocket to receive an insert that can be chilled or heated, providing a hands-free method for applying temperature therapy to the eye area. The eyepatch body can be made from various materials, including antimicrobial fabrics, and features a pocket accessible via different types of fasteners. Inserts are designed to match the body's shape and may contain a cooling gel mixture or a heat-retaining medium. The eyepatch can be secured to the user's face with either a skin-safe adhesive fastener or an adjustable strap. Two methods of use involve cooling or heating the insert, placing it in the pocket, and securing the eyepatch to the face to deliver the therapeutic effect.

In this manner, the heating or cooling eyepatch device of the present invention accomplishes all the foregoing objectives and provides an improved heating and cooling device for application to a user's eye area. More specifically, the device provides heating or cooling relief to a user's eye area without the need to manually hold the device on said eye area. In addition, the device only covers one eye of the user during use.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a heating or cooling eyepatch device comprised of at least one eyepatch body with at least one pocket that receives at least one insert that can be chilled or heated. As a result, the device provides a hands-free method of applying cold or heat to a user's eye.

The body is further comprised of at least one pocket that can be accessed via unfastening at least one fastener. The pocket receives at least one insert. In one embodiment, the insert can be frozen and/or chilled such that it provides a cooling effect to a user's eye area once the device has been applied to a user's eye area. In this embodiment, the insert may be comprised of a gel or liquid mixture encased within the insert body. In this embodiment, the insert body may be made from a polyethylene, nylon, or vinyl, ensuring it is leak-proof and resistant to punctures.

In a different embodiment, the insert is comprised of a heat-retaining medium. In this embodiment, the insert body may be made from materials such as, but not limited to, cotton, polyester, or neoprene, which provide durability and insulation. In one embodiment, the medium is comprised of an additive such as, but not limited to, an essential oil for additional therapeutic benefits.

In one embodiment, the body attaches to the face/eye area of the user via at least one fastener positioned on the rear surface of the body. The fastener is preferably a skin-safe adhesive fastener that allows the body to adhesively attach to a user's face/eye area. In a different embodiment, the body is secured around a user's head via at least one strap. The strap may be made from any material. In one embodiment, the strap is an elastic strap. In a second embodiment, the length of the strap is adjustable, which can be achieved by at least one adjustment fastener such as, but not limited to, a sliding buckle, hook and loop, snap button, etc.

The present invention is also comprised of a first method of using the device. First, a device is provided comprised of an eyepatch body comprised of a pocket, a fastener, and a rear surface comprised of a fastener, and an insert comprised of an insert body that contains a cooling mixture or a heating medium. Then, the insert can be cooled or heated and placed within the pocket, wherein the pocket can then be closed by securing the fastener. Next, the rear surface of the body can be secured to a user's face and/or eye area via the fastener, which is preferably a skin-safe adhesive, to provide a heating or cooling compress effect on said face and/or eye area.

Accordingly, the heating or cooling eyepatch device of the present invention is particularly advantageous as it provides an improved heating and cooling device for application to a user's eye area. More specifically, the device provides heating or cooling relief to a user's eye area without the need to manually hold the device on said eye area. In addition, the device only covers one eye of the user during use. In this manner, the heating or cooling eyepatch device overcomes the limitations of existing heating and cooling devices known in the art used for application to an individual's eye area.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front view of one potential embodiment of a heating or cooling eyepatch device of the present invention in accordance with the disclosed architecture;

FIG. 2 illustrates a front view of two potential embodiment of inserts of a heating or cooling eyepatch device of the present invention in accordance with the disclosed architecture;

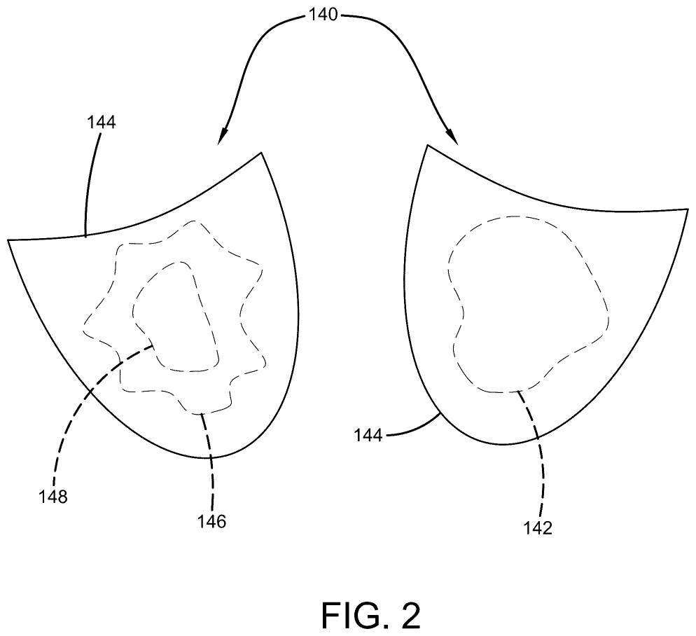

FIG. 3 illustrates a rear view of one potential embodiment of inserts of a heating or cooling eyepatch device of the present invention in accordance with the disclosed architecture;

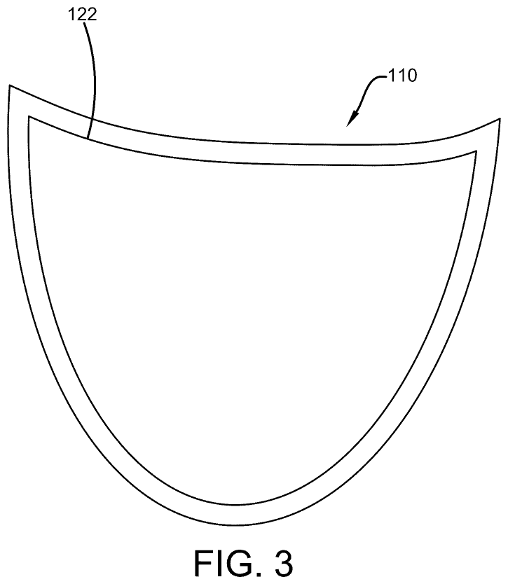

FIG. 4 illustrates a front view of one potential embodiment of a heating or cooling eyepatch device of the present invention in accordance with the disclosed architecture;

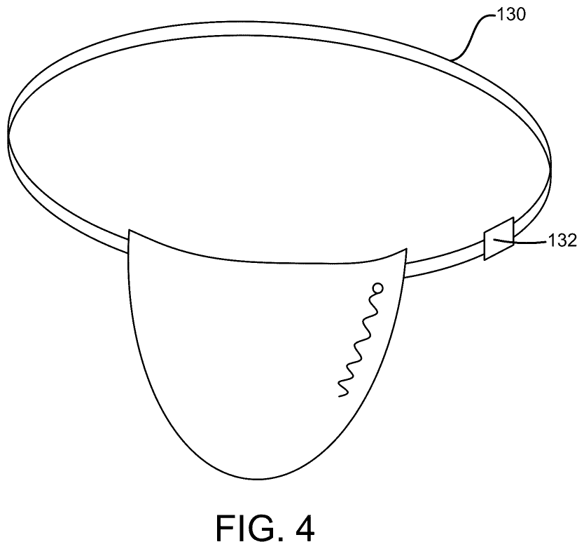

FIG. 5 illustrates a flowchart of a method of using one potential embodiment of a heating or cooling eyepatch device of the present invention in accordance with the disclosed architecture; and

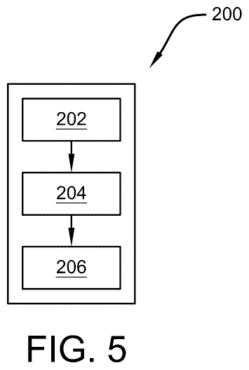

FIG. 6 illustrates a flowchart of a method of using one potential embodiment of a heating or cooling eyepatch device of the present invention in accordance with the disclosed architecture.

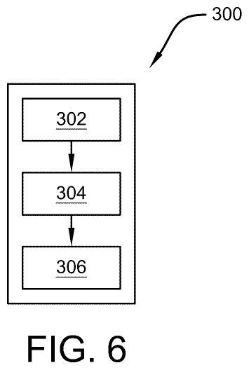

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for an improved heating and cooling device for application to a user's eye area. There also exists a long-felt need in the art for a heating or cooling eyepatch device that provides heating or cooling relief to a user's eye area. More specifically, there exists a long-felt need in the art for a heating or cooling eyepatch device that provides heating or cooling relief to a user's eye area without the need to manually hold the device on said eye area. In addition, there exists a long-felt need in the art for a heating or cooling eyepatch device that provides heating or cooling relief to a user's eye area without the need to manually hold the device on said eye area, wherein the device only covers one eye of the user during use.

The present invention, in one exemplary embodiment, is comprised of a heating or cooling eyepatch device. The device is comprised of a heating or cooling eyepatch device comprised of at least one eyepatch body with at least one pocket that receives at least one insert that can be chilled or heated. As a result, the device provides a hands-free method of applying cold or heat to a user's eye.

The body is further comprised of at least one pocket that can be accessed via unfastening at least one fastener. The pocket receives at least one insert that can be frozen and/or chilled such that it provides a cooling effect to a user's eye area once the device has been applied to a user's eye area. In this embodiment, the insert may be comprised of a gel or liquid mixture encased within the insert body. In this embodiment, the insert body may be made from a polyethylene, nylon, or vinyl, ensuring it is leak-proof and resistant to punctures.

In a different embodiment, the insert is comprised of a heat-retaining medium and may be made from materials such as, but not limited to, cotton, polyester, or neoprene, which provide durability and insulation. In one embodiment, the medium is comprised of an additive such as, but not limited to, an essential oil for additional therapeutic benefits.

In one embodiment, the body attaches to the face/eye area of the user via at least one fastener positioned on the rear surface of the body. The fastener is preferably a skin-safe adhesive fastener that allows the body to adhesively attach to a user's face/eye area. In a different embodiment, the body is secured around a user's head via at least one strap. The strap may be made from any material. In one embodiment, the strap is an elastic strap. In a second embodiment, the length of the strap is adjustable, which can be achieved by at least one adjustment fastener such as, but not limited to, a sliding buckle, hook and loop, snap button, etc.

The present invention is also comprised of a first method of using the device. First, a device is provided comprised of an eyepatch body comprised of a pocket, a fastener, and a rear surface comprised of a fastener, and an insert comprised of an insert body that contains a cooling mixture or a heating medium. Then, the insert can be cooled or heated and placed within the pocket, wherein the pocket can then be closed by securing the fastener. Next, the rear surface of the body can be secured to a user's face and/or eye area via the fastener, which is preferably a skin-safe adhesive, to provide a heating or cooling compress effect on said face and/or eye area.

Accordingly, the heating or cooling eyepatch device of the present invention is particularly advantageous as it provides an improved heating and cooling device for application to a user's eye area. More specifically, the device provides heating or cooling relief to a user's eye area without the need to manually hold the device on said eye area. In addition, the device only covers one eye of the user during use. In this manner, the heating or cooling eyepatch device overcomes the limitations of existing heating and cooling devices known in the art used for application to an individual's eye area.

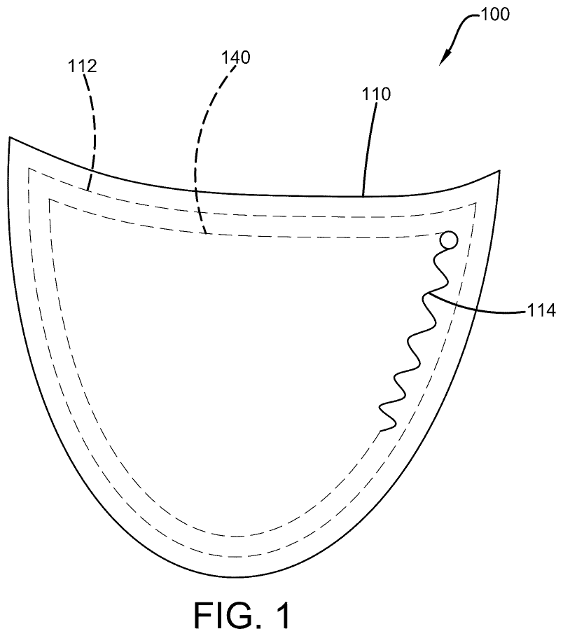

Referring initially to the drawings, FIG. 1 illustrates a front view of one potential embodiment of a heating or cooling eyepatch device 100 of the present invention in accordance with the disclosed architecture. The device 100 is comprised of at least one eyepatch body 110 with at least one pocket 112 that receives at least one insert 140 that can be chilled or heated. As a result, the device 100 provides a hands-free method of applying cold or heat to a user's eye.

The eyepatch body 110 may be any shape and size in different embodiments. The body 110 may be concave or convex in shape. The body 110 may be made from a material such as, but not limited to, a fabric, a leather, a plastic, or a foam material. In one embodiment, the fabric material is anti-microbial. The body 110 is further comprised of at least one pocket 112. The Pocket 112 can be accessed via unfastening at least one fastener 114. The fastener 114 may be any fastener type, such as, but not limited to, a zipper, a hook and loop fastener, a magnet, a snap button, a tie fastener, etc.

The pocket 112 receives at least one insert 140. The insert 140 may be any shape but is preferably the same or similar shape as the body 110. In one embodiment, the insert 140 can be frozen and/or chilled such that it provides a cooling effect to a user's eye area once the device 100 has been applied to a user's eye area. In this embodiment, the insert 140 may be comprised of a gel or liquid mixture 142 encased within the insert body 144. The mixture 142 may be comprised of any combination of water, a thickening agent such as propylene glycol or silica gel, and other compounds like cellulose, sodium polyacrylate, or hydroxyethyl cellulose to enhance viscosity and cooling duration, as well as an anti-microbial agent to prevent bacterial growth. In this embodiment, the insert body 144 may be made from a polyethylene, nylon, or vinyl, ensuring it is leak-proof and resistant to punctures.

In a different embodiment, the insert is comprised of a heat-retaining medium 146. This includes, but is not limited to, any mixture of a gel, a saline solution, or granular materials like a rice, a wheat, or a flaxseed, or a sodium acetate-which crystallizes to release heat when triggered. In this embodiment, the insert body 144 may be made from materials such as, but not limited to, cotton, polyester, or neoprene, which provide durability and insulation. In one embodiment, the medium 146 is comprised of an additive 148 such as, but not limited to, an essential oil for additional therapeutic benefits.

In one embodiment, the body 110 attaches to the face/eye area of the user via at least one fastener 122 positioned on the rear surface 120 of the body 110. The fastener 122 is preferably a skin-safe adhesive fastener that allows the body 110 to adhesively attach to a user's face/eye area. The fastener 122 is preferably positioned around the perimeter of the rear surface 120. In a different embodiment, the body 110 is secured around a user's head via at least one strap 130. The strap 130 may be made from any material. In one embodiment, the strap 130 is an elastic strap. In a second embodiment, the length of the strap 130 is adjustable, which can be achieved by at least one adjustment fastener 132 such as, but not limited to, a sliding buckle, hook and loop, snap button, etc.

The present invention is also comprised of a first method of using 200 the device 100. First, a device 100 is provided comprised of an eyepatch body 110 comprised of a pocket 112, a fastener 114, and a rear surface 120 comprised of a fastener 122, and an insert 140 comprised of an insert body 144 that contains a cooling mixture 142 or a heating medium 146 [Step 202]. Then, the insert 140 can be cooled or heated and placed within the pocket 112, wherein the pocket 112 can then be closed by securing the fastener 114 [Step 204]. Next, the rear surface 120 of the body 110 can be secured to a user's face and/or eye area via the fastener 122, which is preferably a skin-safe adhesive, to provide a heating or cooling compress effect on said face and/or eye area [Step 206].

The present invention is also comprised of a second method of using 300 the device 100. First, a device 100 is provided comprised of an eyepatch body 110 comprised of a pocket 112, a fastener 114, and a strap 130, and an insert 140 comprised of an insert body 144 that contains a cooling mixture 142 or a heating medium 146 [Step 302]. Then, the insert 140 can be cooled or heated and placed within the pocket 112, wherein the pocket 112 can then be closed by securing the fastener 114 [Step 304]. Next, the strap 130 can be placed around a user's head (and may be adjusted via at least one adjustment fastener 132) such that the body 110 contacts a face and/or eye area of a user and provides a heating or cooling compress effect on said face and/or eye area [Step 306].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “heating or cooling eyepatch device” and “device” are interchangeable and refer to the heating or cooling eyepatch device 100 of the present invention.

Notwithstanding the foregoing, the heating or cooling eyepatch device 100 of the present invention and its various components can be of any suitable size and configuration as is known in the art without affecting the overall concept of the invention, provided that they accomplish the above-stated objectives. One of ordinary skill in the art will appreciate that the size, configuration, and material of the heating or cooling eyepatch device 100 as shown in the FIGS. are for illustrative purposes only, and that many other sizes and shapes of the heating or cooling eyepatch device 100 are well within the scope of the present disclosure. Although the dimensions of the heating or cooling eyepatch device 100 are important design parameters for user convenience, the heating or cooling eyepatch device 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A heating or cooling eyepatch device comprising:
an insert; and
an eyepatch body comprised of:
a pocket comprised of a first fastener, wherein the insert is positioned within the pocket; and
a rear surface comprised of a second fastener.

2. The heating or cooling eyepatch device of claim 1, wherein the insert is comprised of a gel or a liquid mixture.

3. The heating or cooling eyepatch device of claim 2, wherein the gel or liquid mixture is comprised of a water, a thickening agent, a cellulose, a sodium polyacrylate, a hydroxyethyl cellulose, and an anti-microbial agent.

4. The heating or cooling eyepatch device of claim 1, wherein the insert is comprised of a heat-retaining medium.

5. The heating or cooling eyepatch device of claim 4, wherein the heat-retaining medium is comprised of a gel, a saline solution, a granular material, or a sodium acetate.

6. The heating or cooling eyepatch device of claim 5, wherein the granular material is comprised of rice, a wheat, or a flaxseed.

7. The heating or cooling eyepatch device of claim 4, wherein a body of the insert is comprised of a cotton, a polyester, or a neoprene.

8. The heating or cooling eyepatch device of claim 4, wherein the heat-retaining medium is comprised of an essential oil.

9. The heating or cooling eyepatch device of claim 1, wherein the second fastener is comprised of a skin-safe adhesive.

10. The heating or cooling eyepatch device of claim 1, wherein the second fastener is positioned around a perimeter of the rear surface.

11. A heating or cooling eyepatch device comprising:
an insert; and
an eyepatch body comprised of:
a pocket comprised of a first fastener, wherein the insert is positioned within the pocket; and
a strap.

12. The heating or cooling eyepatch device of claim 11, wherein the strap is comprised of an adjustable length strap.

13. The heating or cooling eyepatch device of claim 11, wherein the strap is comprised of an adjustment fastener.

14. The heating or cooling eyepatch device of claim 13, wherein the adjustment fastener is comprised of a sliding buckle, a hook and loop, or a snap button.

15. The heating or cooling eyepatch device of claim 11, wherein the insert is comprised of a cooling insert.

16. The heating or cooling eyepatch device of claim 11, wherein the insert is comprised of a heating insert.

17. The heating or cooling eyepatch device of claim 11, wherein the strap is comprised of an elastic strap.

18. The heating or cooling eyepatch device of claim 11, wherein the eyepatch body is comprised of a fabric, a leather, a plastic, or a foam material.

19. A method of using a heating or cooling eyepatch device, the method comprising:
providing a heating or cooling eyepatch device comprised of an eyepatch body comprised of a pocket, a first fastener, and a rear surface comprised of a second fastener, and an insert comprised of an insert body that contains a cooling mixture or a heating medium;
cooling or heating the insert and placing the insert within the pocket, wherein the pocket can be closed via securing the first fastener; and
securing the rear surface to an eye area of a user's face via the second fastener.

20. The method of using a heating or cooling eyepatch device of claim 19, wherein the second fastener is comprised of a skin-safe adhesive.
